(19) United States
(12) Patent Application Publication
(10) Pub. No.: US 2017/0258373 A1
(43) Pub. Date: Sep. 14, 2017

(54) MUSCLE FATIGUE MONITORING SYSTEM

(71) Applicant: Yingxiu PAN, Shenzhen (CN)

(72) Inventor: Yingxiu PAN, Shenzhen (CN)

(73) Assignee: YINGXIUKE LIMITED, Hong Kong (HK)

(21) Appl. No.: 15/607,448

(22) Filed: May 27, 2017

(51) Int. Cl. A61B 5/11; G06F 3/023; A61B 5/22

(52) U.S. Cl. CPC A61B 5/1107; A61B 5/224; G06F 3/023; A61B 5/103

(57) ABSTRACT

A muscle fatigue monitoring system includes an sEMG amplifier module configured to receive sEMG signals and amplify the received sEMG signals; a filter module connected with the sEMG amplifier module; a bit-stream converter connected with the filter module and configured to digitize the sEMG signals and convert the sEMG signals to a discrete signal based on a single threshold without digitizing the complete sEMG signals, and a bit-stream cross correlator connected with the bit-stream converter, the bit-stream cross correlator including a plurality of correlation stages connected in series, a plurality of counters connected with the correlation stages respectively, and a maximum value selector connected to the counters, and configured to continuously correlate the sEMG signals in a given time window, count all time instances where the sEMG signals are the same, compares all the counters in cycles, and find distance between specific reference points on the sEMG signals through the counter with a maximum value.

### FIELD OF THE PATENT APPLICATION

The present patent application generally relates to medical electronics and more specifically to a muscle fatigue monitoring system.

### BACKGROUND

Muscle Fiber Conduction Velocity (MFCV) is a measure of the travelling speed of MUAPs in muscle tissue and is one of the most important items which reflects muscular activity. MFCV can provide a more detailed insight into muscle fatigue and muscle recovery than Power Spectral Density (PSD) monitoring alone. MFCV monitoring would result in better muscle fatigue tracking than Median/Mean frequency analysis.

One conventional method of tracking MFCV is extracting information from one detected sEMG signal alone. Typically, spectral analysis tools are required. The method is sensitive to noise and introduces large variance in the result. Another conventional method is comparing two or more detected sEMG signals along the muscle fiber direction. The electrodes are placed perpendicular to the underlying muscle fibers. Algorithms following this approach include finding the distance between reference points. This method assumes that the two detected signals are identical with the addition of noise. Thus, the two signals would have the same shape with the introduction of a delay. As a result, any specific reference point such as a valley, a peak or a zero can be used to align the two signals and estimate the delay between them. Hence the phase difference between the detected signals can be calculated to estimate MFCV. The time lag at which the cross-correlation function is maximum can be used as an estimator of delay.

CMOS based System-on-Chip (SoC) solutions show significant promise to create solutions for wearable medical devices with small form factor, low power consumption and increased accuracy. Therefore, it is desired to implement the cross-correlation method using low power digital CMOS logic with low computational complexity, high efficiency and good noise immunity.

### SUMMARY

The present patent application is directed to a muscle fatigue monitoring system. In one aspect, a muscle fatigue monitoring system includes an sEMG amplifier module configured to receive sEMG signals and amplify the received sEMG signals; a filter module connected with the sEMG amplifier module; a bit-stream converter connected with the filter module and configured to digitize the sEMG signals and convert the sEMG signals to a discrete signal based on a single threshold without digitizing the complete sEMG signals; a bit-stream cross correlator connected with the bit-stream converter, the bit-stream cross correlator including a plurality of correlation stages connected in series, a plurality of counters connected with the correlation stages respectively, and a maximum value selector connected to the counters, and configured to continuously correlate the sEMG signals in a given time window, count all time instances where the sEMG signals are the same, compares all the counters in cycles, and find distance between specific reference points on the sEMG signals through the counter with a maximum value; a bias generator; a timing control module connected with the bit-stream cross correlator; and a serial peripheral interface connected with the timing control module and the maximum value selector. The sEMG amplifier module includes a plurality of dual channel instrumentation amplifiers and an external floating high-pass filter. The filter module includes two low-pass filters and is configured to extract signal attributes in a frequency band of 10 Hz-500 Hz. The bit-stream converter includes two analog comparators. The maximum value selector includes a plurality of comparing blocks, and is configured to start by including values of the counters in pairs and then proceed with evaluating results of the previous comparisons, each including block being configured to compare two 14 bit numbers. Each correlation stage includes a delay block, a counter and a correlator. The delay block is a D-type flip flop, delay time of the delay block being controlled by a sampling frequency of the system. The counter of each correlation stage is a 14 bit ripple counter with a counter size being selected by analyzing retrospective sEMG data, and the correlator includes a XNOR gate and an AND gate connected with the XNOR gate.

The low-pass filters may be Sallen Key low-pass filters with cutoff frequency of 2.5 kHz.

Reference voltages of the two analog comparators may be kept separate to allow offset mismatch compensation.

In another aspect, a muscle fatigue monitoring system includes an sEMG amplifier module configured to receive sEMG signals and amplify the received sEMG signals; a filter module connected with the sEMG amplifier module; a bit-stream converter connected with the filter module and configured to digitize the sEMG signals and convert the sEMG signals to a discrete signal based on a single threshold without digitizing the complete sEMG signals, and a bit-stream cross correlator connected with the bit-stream converter, the bit-stream cross correlator including a plurality of correlation stages connected in series, a plurality of counters connected with the correlation stages respectively, and a maximum value selector connected to the counters, and configured to continuously correlate the sEMG signals in a given time window, count all time instances where the sEMG signals are the same, compares all the counters in cycles, and find distance between specific reference points on the sEMG signals through the counter with a maximum value. The sEMG amplifier module includes a plurality of dual channel instrumentation amplifiers and an external floating high-pass filter. The filter module includes two low-pass filters. The bit-stream converter includes two analog comparators, and each correlation stage includes a delay block, a counter and a correlator.

The muscle fatigue monitoring system may further including a bias generator, a timing control module connected with the bit-stream correlator, and a serial peripheral interface connected with the timing control module and the maximum value selector.

The filter module includes may be configured to extract signal attributes in a frequency band of 10 Hz-500 Hz.

The maximum value selector may include a plurality of comparing blocks, and may be configured to start by comparing values of the counters in pairs and then proceed with evaluating results of the previous comparisons, each comparing block being configured to compare two 14 bit numbers.

The delay block may be a D-type flip flop, delay time of the delay block may be controlled by a sampling frequency of the system.

The counter of each correlation stage may be a 14 bit ripple counter with a counter size being selected by analyzing retrospective sEMG data.

The correlator includes a XNOR gate and an AND gate connected with the XNOR gate.

### BRIEF DESCRIPTION OF THE DRAWINGS

FIG. 1 is a block diagram of a muscle fatigue monitoring system in accordance with an embodiment of the present patent application.

FIG. 2 illustrates the bit-stream cross correlator of the muscle fatigue monitoring system as depicted in FIG. 1.

FIG. 3 illustrates the correlation stages as depicted in FIG. 2.

FIG. 4 illustrates the sequential logic that the maximum value selector as depicted in FIG. 2 uses.

### DETAILED DESCRIPTION

Reference will now be made in detail to a preferred embodiment of the muscle fatigue monitoring system disclosed in the present patent application, examples of which are also provided in the following description. Exemplary embodiments of the muscle fatigue monitoring system disclosed in the present patent application are described in detail, although it will be apparent to those skilled in the relevant art that some features that are not particularly important to an understanding of the muscle fatigue monitoring system may not be shown for the sake of clarity.

Furthermore, it should be understood that the muscle fatigue monitoring system disclosed in the present patent application is not limited to the precise embodiments described below and that various changes and modifications thereof may be effected by one skilled in the art without departing from the spirit or scope of the protection. For example, devices and/or features of different illustrative embodiments may be combined with each other and/or substituted for each other within the scope of this disclosure.

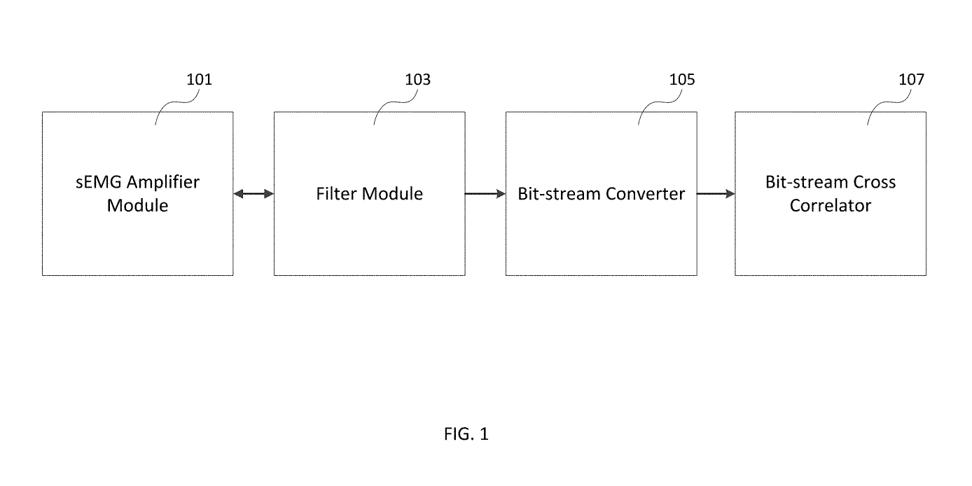

FIG. 1 is a block diagram of a muscle fatigue monitoring system in accordance with an embodiment of the present patent application. Referring to FIG. 1, the muscle fatigue monitoring system includes an sEMG amplifier module 101, a filter module 103 connected with the sEMG amplifier module 101, a bit-stream converter 105 connected with the filter module 103, and a bit-stream cross correlator 107 connected with the bit-stream converter 105.

The sEMG amplifier module 101 includes a plurality of dual channel instrumentation amplifiers and is configured to receive sEMG signals and amplify the received sEMG signals. The sEMG amplifier module 101 is capable of rejecting up to 300 mV DC Polarization Voltage (PV) from the bio-potential electrodes.

The sEMG amplifier module 101 further includes an external floating high-pass filter. Compared to using conventional passive high-pass filters, no grounded resistors are required, which result in very large common mode input impedance.

The filter module 103 includes two low-pass filters and is configured to extract signal attributes in a frequency band of 10 Hz-500 Hz. Preferably the low-pass filters are Sallen Key low-pass filters with cutoff frequency of 2.5 kHz.

The bit-stream converter 105 includes two analog comparators and is configured to digitize the sEMG signals. The reference voltages of the two comparators are kept separate to allow offset mismatch compensation.

The bit-stream cross correlator 107 is configured to continuously correlate the sEMG signals in a given time window, count all time instances where the sEMG signals are the same, compares all the counters in cycles, and find distance between specific reference points on the sEMG signals through the counter with a maximum value.

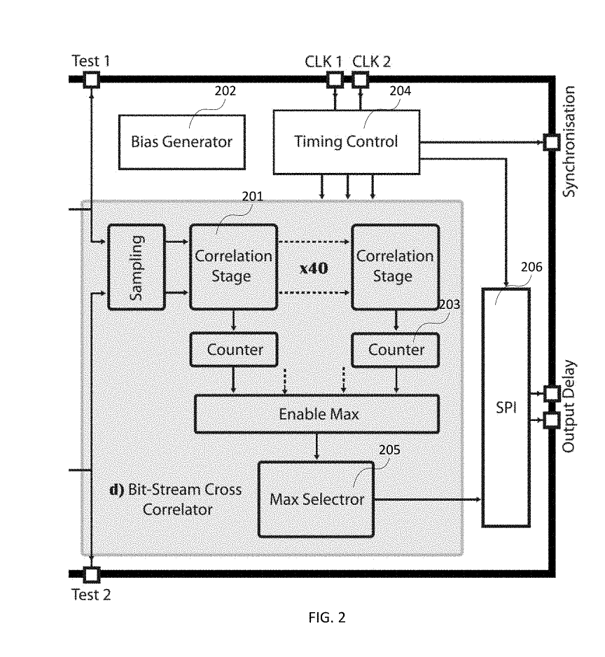

FIG. 2 illustrates the bit-stream cross correlator 107 of the muscle fatigue monitoring system depicted in FIG. 1. Referring to FIG. 2, the bit-stream cross correlator 107 includes a plurality of correlation stages 201 connected in series. The bit-stream cross correlator 107 further includes a plurality of counters 203 connected with the correlation stages 201 respectively. At the end of the correlation time window, all the counters 203 of the system are read. The correlation stage (i.e. delay) of the counter with the maximum value best represents the time lag between the two input signals.

Referring to FIG. 2, the bit-stream cross correlator 107 further includes a maximum value selector 205 connected to the counters 203 and configured to compares all the counters 203 in cycles. The maximum value selector 205 includes a number of comparing blocks, each comparing block being configured to compare two 14 bit numbers. The maximum value selector 205 starts by comparing all the results (i.e. values of the counters) in pairs and then proceeds with evaluating the results of the previous comparisons.

Referring to FIG. 1 and FIG. 2, the muscle fatigue monitoring system further includes a bias generator 202, a timing control module 204 connected with the bit-stream correlator 107, and a Serial Peripheral Interface (SPI) connected with the timing control module 204 and the maximum value selector 205.

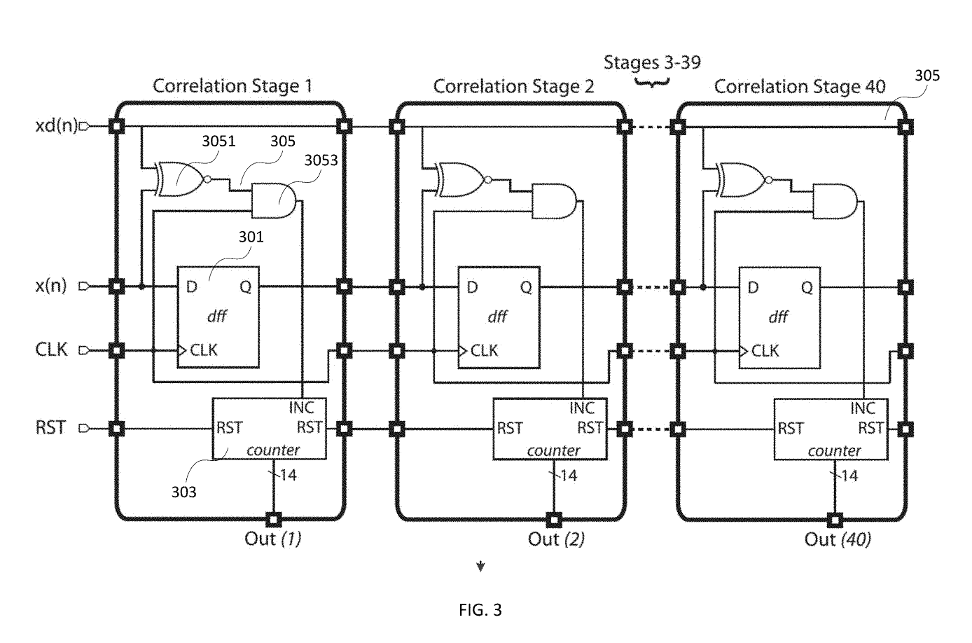

FIG. 3 illustrates the correlation stages depicted in FIG. 2. Referring to FIG. 2 and FIG. 3, each correlation stage 201 includes a delay block 301, a counter 303 and a correlator 305. In this embodiment, the delay block 301 is a D-type flip flop. The delay time is controlled by the sampling frequency of the system. The counter 303 is a 14 bit ripple counter. The counter size was selected by analyzing retrospective sEMG data to allow operation with correlation time windows over 1 second and high sampling frequencies. The correlator 305 includes a XNOR gate 3051 and an AND gate 3053 connected with the XNOR gate 3051. The XNOR gate 3051 is used as a bit correlator, which improves the conventional AND gate design by taking all possible digital cases into consideration.

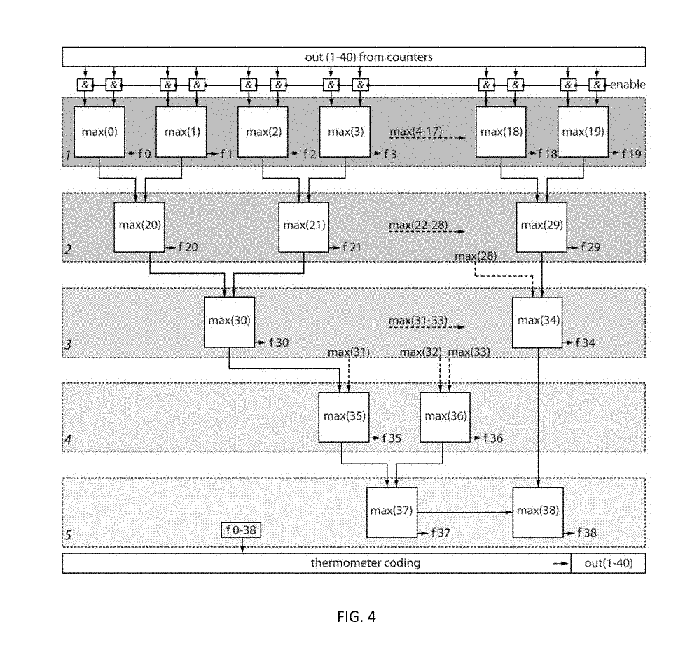

FIG. 4 illustrates the sequential logic that the maximum value selector 205 as depicted in FIG. 2 uses. Referring to FIG. 4, every maximum operation returns a binary flag, which passes down to the next comparison and indicates which one of the two compared numbers is the maximum. A binary one means the first of the two numbers is bigger. The counter position number (delay number) and not the counter value is returned when the operation is finished.

In this embodiment, the bit-stream cross correlator 107 is configured to execute a cross-correlation algorithm and compute the time delay between the sEMG signals. The algorithm can be applied to finding the distance between specific reference points such as a valley, a peak or a zero, so that the cross correlation process is simplified. The sEMG signals are converted by the bit-stream converter to a discrete signal based on a single threshold, without digitizing the complete sEMG signals, while retaining the necessary information for cross correlation and delay estimation. This eliminates the need to cross-correlating the whole sEMG signal, while only a single bit approximation of the sEMG signals is required to be cross-correlated, so that the cross-correlator's architecture is greatly simplified.

In this embodiment, bit-stream buffer window is eliminated by continuously cross correlating the two sEMG signals in a given time window. This is achieved by counting all the time instances where the two signals are the same. A cross correlation time window replaces the buffer window for x(n).

In this embodiment, discrete time lags for the cross-correlation output are obtained by continuously delaying the input signal. Cross correlation result for every discrete time delay is obtained. The time lag between the two signals is returned by the counter with the larger value, so that the number of transistors required is greatly reduced.

While the present patent application has been shown and described with particular references to a number of embodiments thereof, it should be noted that various other changes or modifications may be made without departing from the scope of the present invention.

What is claimed is:

1. A muscle fatigue monitoring system comprising:
an sEMG amplifier module configured to receive sEMG signals and amplify the received sEMG signals;
a filter module connected with the sEMG amplifier module;
a bit-stream converter connected with the filter module and configured to digitize the sEMG signals and convert the sEMG signals to a discrete signal based on a single threshold without digitizing the complete sEMG signals;
a bit-stream cross correlator connected with the bit-stream converter, the bit-stream cross correlator comprising a plurality of correlation stages connected in series, a plurality of counters connected with the correlation stages respectively, and a maximum value selector connected to the counters, and configured to continuously correlate the sEMG signals in a given time window, count all time instances where the sEMG signals are the same, compares all the counters in cycles, and find distance between specific reference points on the sEMG signals through the counter with a maximum value;
a bias generator;
a timing control module connected with the bit-stream cross correlator; and
a serial peripheral interface connected with the timing control module and the maximum value selector; wherein:
the sEMG amplifier module comprises a plurality of dual channel instrumentation amplifiers and an external floating high-pass filter;
the filter module comprises two low-pass filters and is configured to extract signal attributes in a frequency band of 10 Hz-500 Hz;
the bit-stream converter comprises two analog comparators;
the maximum value selector comprises a plurality of comparing blocks, and is configured to start by comparing values of the counters in pairs and then proceed with evaluating results of the previous comparisons, each comparing block being configured to compare two 14 bit numbers;
each correlation stage comprises a delay block, a counter and a correlator;
the delay block is a D-type flip flop, delay time of the delay block being controlled by a sampling frequency of the system;
the counter of each correlation stage is a 14 bit ripple counter with a counter size being selected by analyzing retrospective sEMG data; and
the correlator comprises a XNOR gate and an AND gate connected with the XNOR gate.

2. The muscle fatigue monitoring system of claim 1, wherein the low-pass filters are Sallen Key low-pass filters with cutoff frequency of 2.5 kHz.

3. The muscle fatigue monitoring system of claim 1, wherein reference voltages of the two analog comparators are kept separate to allow offset mismatch compensation.

4. A muscle fatigue monitoring system comprising:
an sEMG amplifier module configured to receive sEMG signals and amplify the received sEMG signals;
a filter module connected with the sEMG amplifier module;
a bit-stream converter connected with the filter module and configured to digitize the sEMG signals and convert the sEMG signals to a discrete signal based on a single threshold without digitizing the complete sEMG signals; and
a bit-stream cross correlator connected with the bit-stream converter, the bit-stream cross correlator comprising a plurality of correlation stages connected in series, a plurality of counters connected with the correlation stages respectively, and a maximum value selector connected to the counters, and configured to continuously correlate the sEMG signals in a given time window, count all time instances where the sEMG signals are the same, compares all the counters in cycles, and find distance between specific reference points on the sEMG signals through the counter with a maximum value; wherein:
the sEMG amplifier module comprises a plurality of dual channel instrumentation amplifiers and an external floating high-pass filter;
the filter module comprises two low-pass filters;
the bit-stream converter comprises two analog comparators; and
each correlation stage comprises a delay block, a counter and a correlator.

5. The muscle fatigue monitoring system of claim 4 further comprising a bias generator, a timing control module connected with the bit-stream correlator, and a serial peripheral interface connected with the timing control module and the maximum value selector.

6. The muscle fatigue monitoring system of claim 4, wherein the filter module comprises is configured to extract signal attributes in a frequency band of 10 Hz-500 Hz.

7. The muscle fatigue monitoring system of claim 4, wherein the maximum value selector comprises a plurality of comparing blocks, and is configured to start by comparing values of the counters in pairs and then proceed with evaluating results of the previous comparisons, each comparing block being configured to compare two 14 bit numbers.

8. The muscle fatigue monitoring system of claim 4, wherein the delay block is a D-type flip flop, delay time of the delay block being controlled by a sampling frequency of the system.

9. The muscle fatigue monitoring system of claim 4, wherein the counter of each correlation stage is a 14 bit ripple counter with a counter size being selected by analyzing retrospective sEMG data.

10. The muscle fatigue monitoring system of claim 4, wherein the correlator comprises a XNOR gate and an AND gate connected with the XNOR gate.
